(19) United States
(12) Patent Application Publication
(10) Pub. No.: US 2025/0025131 A1
(43) Pub. Date: Jan. 23, 2025

(54) ULTRASOUND IMAGING USING C-WAVE BEAMS FOR INCREASING FRAME RATE AND SIGNAL STRENGTH

(71) Applicant: CLOUDSTREAM MEDICAL IMAGING, INC., Houston, TX (US)

(72) Inventors: Chengbin PENG, Houston, TX (US); Jun TANG, Houston, TX (US)

(21) Appl. No.: 18/713,596

(22) PCT Filed: Dec. 8, 2022
(86) PCT No.: PCT/US2022/052198

§ 371 Date: May 24, 2024

Related U.S. Application Data
(60) Provisional application No. 63/289,100, filed on Dec. 13, 2021.

(51) Int. Cl. A61B 8/00; G01S 7/52; G01S 15/89

(52) U.S. Cl. CPC A61B 8/4488; A61B 8/461; A61B 8/469; G01S 7/5202; G01S 15/8915

(57) ABSTRACT

A method of acquiring ultrasound radio-frequency (RF) data using C-wave beams includes: providing an ultrasound transducer, the ultrasound transducer including a plurality of elements acting as both transmitters and receivers; transmitting sound waves from the transmitters of the ultrasound transducer within a transmit aperture with transmitter time delays being programed in such a way that sound waves are the C-wave beams that bend inward on both edges in a C shape; and receiving the sound waves using the receivers of the ultrasound transducer. The coherent wavefront includes a variable tilt angle and a variable apex, and the variable apex moves away from a center of the ultrasound transducer as the variable tilt angle increases in absolute value. A system for acquiring and processing ultrasound radio-frequency (RF) data using C-wave beams is also disclosed.

This application claims priority to U.S. Provisional Patent Application No. 63/289,100, filed on Dec. 13, 2021, which is incorporated by reference for all purposes as if fully set forth herein.

### FIELD OF THE INVENTION

The present invention relates to a method of acquiring ultrasound radio-frequency (RF) data using C-wave beams and a system for acquiring and processing ultrasound radio-frequency (RF) data using the C-wave beams.

### BACKGROUND OF THE INVENTION

Medical ultrasound imaging for diagnosis has advantages, such as cost, real-time imaging, portability, and its harmless effect, over computerized tomography (CT) and magnetic resonance imaging (MRI) [1, 2]. However, the resolution of the ultrasound imaging system is usually lower than that of CT and MRI systems [3]. Ultrasound imaging technology is progressing towards high quality and sharp resolution, thanks to better data acquisition hardware and sophisticated processing software [4].

Commonly used ultrasound transducers include linear array transducers, curved array transducers, and phased array transducers. Ultrasound images of a linear array transducer have a rectangular shape. Since the linear array is normally used for precise imaging, its operating frequency is high. In contrast, the convex array is used to acquire a wide and deep ultrasound image at the cost of the resolution. For this reason, the elements of the convex array are arranged in a curved fashion along the azimuthal direction. The method of acquiring an image using a convex array is the same as that when using a linear array but the ultrasound image of the convex array has a fan shape. In the case of a target object being behind obstacles it is difficult to obtain an ultrasound image using the linear array or the convex array. For this case, a phased array can be used by steering the ultrasound beams at oblique angles. Ultrasound images of a phased array have a circular cone shape. Recently concave ultrasound transducers are also proposed for 3D arrays [5]. 3D ultrasound imaging systems are in actively development and a lot of innovations are happening in that space [6].

Commonly used ultrasound data acquisition methods for medical applications include focused beams, divergent beams, and planewave beams [7-10]. Single element transmission is seldom used in medical ultrasound applications because it is time consuming for data collection and poor in signal to noise ratio. In ultrasound data acquisition using focused beams the time delay of each transmitter is electronically controlled in such a way that, at the focal point of a beam which is in front of the transducer and inside the image domain, transmitters employed by this beam emit waves that arrive at the focal point at the same time. The in-sonification at the focal point is very strong and it rapidly dies down away from the focal point. In ultrasound data acquisition using divergent beams the time advance of each transmitter is electronically controlled in such a way that, at the focal point of a given beam which is behind the transducer and outside the image domain, transmitters employed by this beam virtually emit waves from the focal point at the same time. The in-sonification in the image domain is weak and divergent out. In ultrasound data acquisition using planewave beams the time advance of each transmitter is similar to that of a divergent beam except the virtual focal point is far away behind the transducer. All transmitters participate in the excitation of each planewave beam. The in-sonification of a planewave beam in the image domain is weak and uniform [1,7]. Most commercial ultrasound scanners employ a focused beam data acquisition because the signal to noise ratio is much higher in the final image. The downside of focused beam data acquisitions is much reduced frame rate compared to the planewave modality [9-10].

The present invention relates to acquisition and processing of ultrasound data for medical applications. In particular, the invention addresses two urgent needs in medical diagnostic imaging: (1) faster frame rate for imaging blood flows and a beating heart (2) accurate detection of the speed and direction of tissue movements that requires high signal to noise ratio [7-10]. We call our invention C-wave beam data acquisition and processing, or C-wave beamforming. We use the two terminologies interchangeably. The C-wave beamforming is as fast as planewave beamforming. It also has higher signal to noise ratio in the center part of its image domain where it is of most interest to a physician, thanks to its ability to direct energies towards the center for all beams. The ability to rapidly illuminate a large volume of tissues with ultrasound in-sonification, especially at the center part with stronger focusing capability, and properly image all echoes reflected from acoustic contrasts in the tissues makes the C-wave beamforming a useful tool for diagnosing cardiovascular diseases, heart diseases, blood blockages, malignant cancers where blood flows are faster and plenty, to name a few. It has potential to replace planewave modality.

### SUMMARY OF THE INVENTION

In one embodiment, the present application discloses a method of acquiring ultrasound radio-frequency (RF) data using C-wave beams. The method includes: providing an ultrasound transducer, the ultrasound transducer including a plurality of elements acting as both transmitters and receivers; transmitting sound waves from the transmitters of the ultrasound transducer within a transmit aperture with transmitter time delays being programed in such a way that sound waves are the C-wave beams that bend inward on both edges in a C shape; and receiving the sound waves using the receivers of the ultrasound transducer. The coherent wavefront includes a variable tilt angle and a variable apex, and the variable apex moves away from a center of the ultrasound transducer as the variable tilt angle increases in absolute value; the variable apex is an acoustical energy focusing center of the coherent wave; and the variable tilt angle is an angle between a line connecting the center of an ellipse of the C-wave wavefront and the center of the ultrasound transducer and a vertical line passing the center of the ultrasound transducer.

In another embodiment, the ultrasound transducer is a linear array transducer, a curved array transducer, a phased array transducer, or a matrix array transducer.

In another embodiment, a first group of the elements of the ultrasound transducer transmit a first local coherent wave propagating in a first inward direction, a second group of elements of the ultrasound transducer transmits a second local coherent wave in a second inward direction; the first inward direction opposes the second inward direction; and the first local coherent wave and second local coherent wave combine to form the C-wave beams.

In another embodiment, the elements at both edges of the ultrasound transducer start transmission earlier than the elements at the center of the ultrasound transducer with a time slope that is a function of the variable tilt angle and the variable apex.

In another embodiment, the absolute value of the tilt angle is equal or greater than 0 and equal or less than a predefined positive number. The predefined positive number can be, for example, 20, 25, 30, 32, 34, 36, 38, 40, 45, 50, 55, 60, 65, 70, 75, 80, 85, or 90.

In another embodiment, the C-wave beams have a 3D bowl shape with two variable tilt angles and one variable apex.

In another embodiment, the method further includes: (i) taking a trace from input data acquired using the C-wave beams; (ii) optionally performing a frequency filtering to protect the trace from aliasing or excessive wavelet distortion during beamforming; (iii) spraying the data samples of the trace along impulse response curves; (iv) accumulating contributions at each image location, optionally forming partial image volumes for generation of common image point gathers; (v) repeating steps (i)-(iv) for all traces in the data; and (vi) performing post processing and coherent compounding to obtain a final image.

In another embodiment, the present application discloses a system for acquiring and processing ultrasound radio-frequency (RF) data acquired using C-wave beams. The system includes: an ultrasound transducer, the ultrasound transducer including a plurality of elements; a transmission and reception device; a display device; a keyboard; a pointing device; and a processing unit that contains a CPU (central processing unit) and a GPU (graphic processing unit). The CPU and the GPU are adapted to: acquire, via the ultrasound transducer and the transmission and reception device, raw RF data using C-wave beams; process and send the raw RF data to CPU memories or GPU memories; beamform the raw RF data on the CPU, the GPU, or both to obtain an ultrasound image; process and send the ultrasound image to the display device; display, via the display device, the ultrasound image; and repeat the above steps for a next frame.

In another embodiment, the display device is connected to the processing unit remotely, via internet connection, wireless connection, or satellite connection.

In another embodiment, the ultrasound transducer is a linear array transducer, a curved array transducer, a phased array transducer, or a matrix array transducer.

In another embodiment, the keyboard is a wireless keyboard or a software keyboard installed on the processing unit.

In another embodiment, the transmission and reception device is programmed to transmit and receive various types of the C-wave beams.

In another embodiment, the pointing device is a touch screen.

In another embodiment, using the C-wave beams includes: providing an ultrasound transducer, the ultrasound transducer including a plurality of elements acting as both transmitters and receivers; transmitting sound waves from the transmitters of the ultrasound transducer within a transmit aperture with transmitter time delays being programed in such a way that sound waves are the C-wave beams that bend inward on both edges in a C shape; and receiving the sound waves using the receivers of the ultrasound transducer. The coherent wavefront includes a variable tilt angle and a variable apex, and the variable apex moves away from a center of the ultrasound transducer as the variable tilt angle increases in absolute value; the variable apex is an acoustical energy focusing center of the coherent wave; and the variable tilt angle is an angle between a line connecting the center of an ellipse of the C-wave wavefront and the center of the ultrasound transducer and a vertical line passing the center of the ultrasound transducer.

In another embodiment, using the C-wave beams includes: making the C-wave beams having a 3D bowl shape with two variable tilt angles and one variable apex.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is an illustration of C-wave ultrasound data acquisition using a 128-element linear array transducer. The horizontal axis is lateral coordinate and the vertical axis is depth. A “C”-shaped wavefront is initiated to propagate inside an image domain, illuminating target objects from both sides. The wavefront can be tilted. Its apex moves laterally away from the probe center as the tilt angle increases.

FIG. 2 shows wavefront diagrams of three C-waves: (left) a negative tilted C-wave (tilt angle α<0), (middle) a symmetric C-wave (tilt angle α=0), and (right) a positive tilted C-wave (tilt angle α>0). In each diagram the solid line from A to B is a wavefront, the arrows are wave propagation directions; and dash line is the trajectory of (X0, Z0) which is the center of an ellipse. α is the tilt angle. Tx aperture is the entire array of a transducer with a total length of L.

FIG. 3 shows a wavefront diagram of a C-wave in 3D: the thick solid line is the wavefront (a bowl in 3D); and dash line is the trajectory of (X0, Z0) which is the center of an ellipsoid of revolution. The tilt of the wavefront is described by two tilt angles in 3D, one is in the x-z plane and another one is in the y-z plane. The apex is the flattest point on the bowl. As the tilt angles become large the apex moves away from the center of a matrix array transducer.

FIG. 4 is an illustration of a transmission pattern of C-wave ultrasound beam data acquisition using a 128-element linear array transducer placed vertically. The horizontal axis is lapse time. To is the time ADC recording is activated, and to is the time the first transmitter is activated. The Tx delay of a transmitter is the difference in time stamps of two events: one for the probe center (PC) and another one for the transmitter. To can be less than, equal to, or great than to.

FIG. 5 is an illustration of a reception pattern for C-wave ultrasound beam data acquisition for a 128-element linear array transducer placed vertically. The horizontal axis is lapse time. The receiving elements are activated with a fixed time delay after the corresponding transmitting elements, and ti is the time the first receiver is activated.

FIG. 6 shows the formation of envelope of a set of ellipses: Each ellipse is an impulse response curve for a single transmitter and a single receiver pair. The beam impulse response curve (thick line) is an impulse response for all transmitters and a single receiver.

FIG. 7 is a workflow diagram of our C-wave beamformer: Each trace of a C-wave beam is beamformed by spraying all samples onto their impulse response curves, contributing to partial image volumes in accordance with an attribute associated with each point on the impulse response curves. The partial image volumes are sorted into common image point gathers. Coherent compounding is used to sum the common image point gathers to form the final image.

FIG. 8 shows a phantom model: white dots are point scatters and white lines are reflectors in the phantom model.

FIG. 9 shows the comparisons of raw data between a planewave beam and a C-wave beam: Left is a planewave beam data with a tilt angle of −5 degree. Right is a C-wave beam data with a tilt angle of −5 degree. All displays are individually normalized.

FIG. 10 shows the comparisons of a planewave image and a C-wave image: Left is the final image of 74 planewaves from −32 degree to +32 degree. Right is the final image of 74 C-waves with tilt angles from −32 degree to +32 degree. All displays are shown in 60 dB.

FIG. 11 includes (Left) an image of 74 planewave beams with −30 dB random noises added to the synthetic data, and (Right) an image of 74 C-wave beams with −30 dB random noises added to the synthetic data. All displays are shown in 60 dB.

FIG. 12 is a schematic representation of the C-wave beam imaging architecture of one embodiment of the present invention.

### DETAILED DESCRIPTION OF THE ILLUSTRATED EMBODIMENTS

Reference will now be made in detail to embodiments of the present invention, example of which is illustrated in the accompanying drawings.

The present invention proposes a novel design for acquiring ultrasound beam data using a linear, curved, phased, or matrix array transducer. In this design all elements on the transducer are used to transmit a coherent wave that bends inward on both edges in a “C” shape, focusing acoustical energies toward the center portion of an image domain. The coherent wavefront has a tilt angle and an apex, with the apex moving away from the probe center as the tilt angle increases. Transmitters at both edges are fired earlier than the transmitter near the apex, with one edge significantly earlier than the other edge depending on the sign of the tilt angle. The C-wave data acquisition is as efficient as a conventional plane wave data acquisition, with better image resolution and signal to noise ratio at the center portion of the image domain. Ultrasound scanners configured with C-wave beam data acquisition and processing are particularly suitable for imaging tissues in motion such as a beating heart and micro vibration of artery walls. They are also suitable for imaging flowing objects such as gas bubbles in a blood stream and rapid blood flows around a malignant cancerous lesion.

A set of ultrasound data is collected with a novel design of transmission pattern of a transducer whose elements are arranged in a linear, curved, phased, or matrix array. We call this C-wave beam data. In this design all elements on the transducer are used to transmit a coherent wave that bends inward on both edges in a “C” shape, focusing acoustical energies toward the center portion of an image domain. The coherent wavefront has a tilt angle and an apex, with the apex moving away from the probe center as the tilt angle increases. Transmitters at both edges are fired earlier than the transmitter near the apex, with one edge significantly earlier than the other edge depending on the sign of the tilt angle. The C-wave data acquisition is as efficient as a conventional plane wave data acquisition, with better image resolution and much improved signal to noise ratio at the center portion of the image domain.

To properly beamform C-wave ultrasound beam data we devise the following special processing steps: (i) take one input trace from a C-wave ultrasound beam data; (ii) optionally perform frequency filtering to protect the data from aliasing or excessive wavelet distortion during beamforming; (iii) spray the data along impulse response curves calculated using equations disclosed in this invention; (iv) accumulate contributions at each image location, optionally form partial image volumes for generation of common image point gathers; (v) repeat steps (i)-(iv) for all data traces of all input beams; and (vi) perform post processing and coherent compounding to obtain the final image.

The C-wave data beamforming is as fast as the planewave data beamforming but with better resolution and signal to noise ratio near the center.

### Technical Description

Focused ultrasound beams are widely used in commercial B-mode diagnostic imaging of tissues and organs [1, 3]. Less common are divergent ultrasound beams and planewave ultrasound beams. Planewave ultrasound beams are particularly promising for its high frame rate and uniform illumination [7-10]. A high frame rate data acquisition is necessary for imaging objects in motion, such as blood flows, beating hearts, and micro vibrations inside tissues. We propose a new data acquisition method that can achieve the same frame rate of a planewave beam data acquisition with better resolution and signal to noise ratio.

### Part I: C-Wave Beam Data Acquisition

### 1.1 Definition of C-Wave Beams

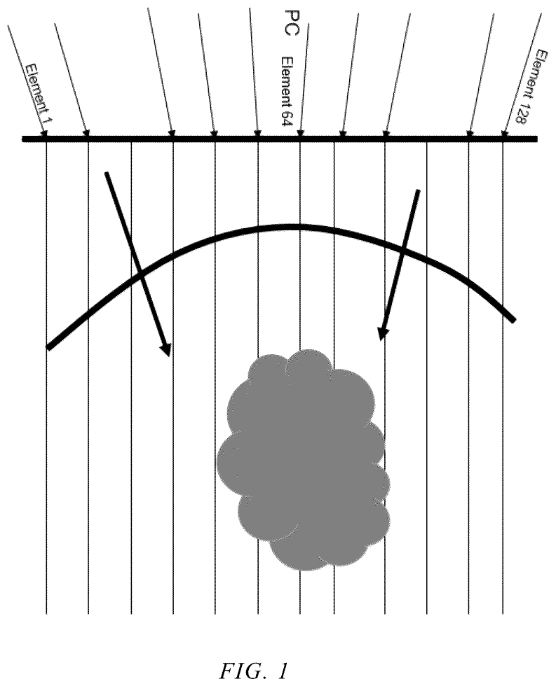

A C-wave ultrasound beam data is collected with a novel design of transmission pattern of a transducer whose elements are arranged in a linear, curved, phased, or matrix array. In this design of transmission pattern all elements on the transducer are used to transmit a coherent wave that bends inward on both edges in a “C” shape, focusing acoustical energies toward the center portion of an image domain (FIG. 1).

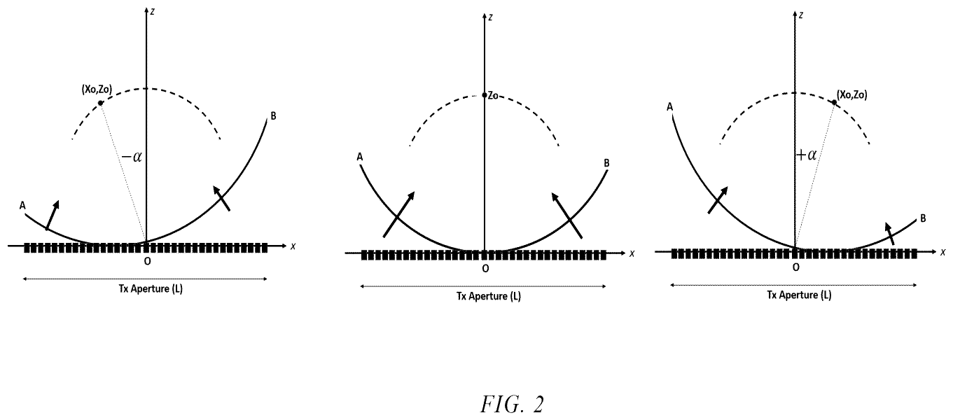

The coherent wavefront has a tilt angle and an apex (FIG. 2), with the apex moving away from the probe center as the tilt angle increases (in absolute value). If the tilt angle is zero, the apex is at the center of the transducer. If the tilt angle is larger than zero, the apex moves to the right and the wavefront tilts to the right. If the tilt angle is less than zero, the apex moves to the left and the wavefront tilts to the left. Transmitters at both edges are fired earlier than the transmitters near the apex, with one edge significantly earlier than the other edge depending on the sign of the tilt angle.

Beams in our C-wave data acquisition always focus acoustic energies towards the center of the image domain, which is distinctly different from beams in plane wave data acquisition. With C-wave data acquisition very little energy is wasted. Majority of transmitted acoustic energies is directed towards tissues under examination. This stronger focusing ability (towards the center) and having a large volume of in-sonification make C-wave beams (1) better than both focused beams and planewave beams, and (2) more desirable for many diagnostic imaging applications, especially for tissues and organs in motion.

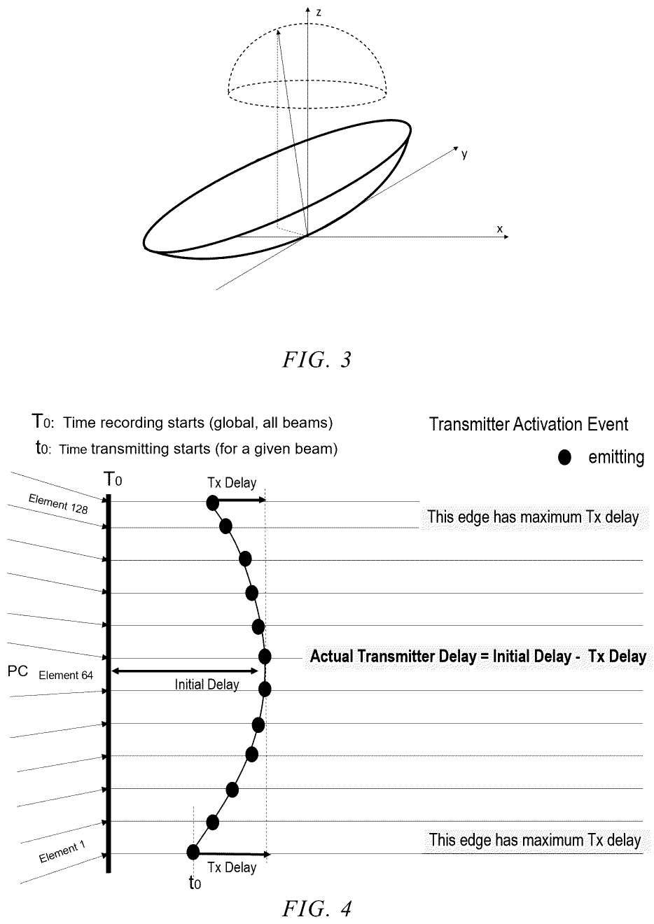

FIG. 3 shows a 3D wavefront of a C-wave beam excited by a matrix array transducer. The wavefront shape is a bowl, an ellipsoid of revolution that can be described by two tilt angles (one in the x-z plane and another in the y-z plane) and an apex. The center of the ellipsoid is located on the dashed sphere far away from the origin. As the wavefront tilts in either direction the apex moves away from the center of the transducer. The 3D C-wave beam can focus energies towards the center from all directions.

### 1.2 C-Wave Transmission Design

FIG. 4 shows the transmission design for acquisition of C-wave ultrasound beam data. The horizontal axis is lapsing time and the vertical axis is element position. We use a linear array of 128 elements as an example. The design equally applies to other array configurations, such as linear arrays with more than or less than 128 elements, curved arrays with arbitrary number of elements, phased arrays with arbitrary number of elements, or matrix arrays with arbitrary number of elements. In FIG. 3 the transducer is placed vertically at the left. Acoustic waves propagate from left to right into human tissues in various tilted angles. The initial delay is set by the acquisition system. The Tx delay pattern is C-shaped, with or without a tilt angle. The total delay for a transmitter is the initial delay minus the Tx delay. The tilt angle α varies from +/− of a pre-defined maximum angle. The Tx delay values are computed using a pre-defined formula that is a function of the tilt angle, the position of a transmitter in the array aperture, and some other parameters. The formula is given in Part II of this application. Other formula can also be used as long as the resulting wavefront can focus energies towards the center of the image domain. In general, as the tilt angle increases the apex of the Tx delay curve moves further away from the center of a transducer.

### 1.3 C-Wave Reception Design

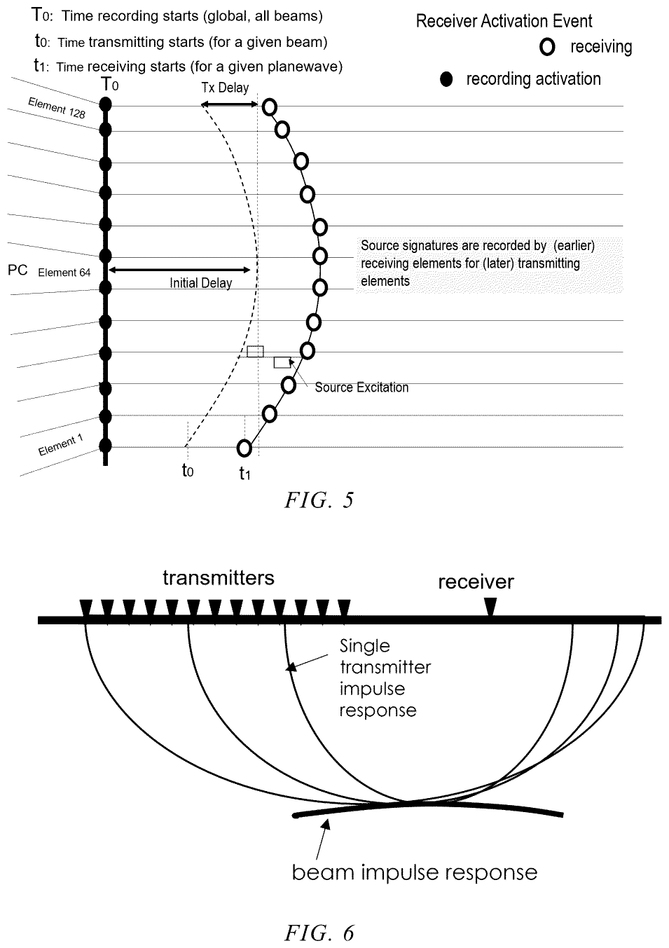

FIG. 5 shows the reception design for acquisition of C-wave ultrasound beam data. The horizontal axis is lapsing time and the vertical axis is element position. We use a linear array of 128 elements as an example. Other array configurations work equally well. The receivers are activated after a fixed time delay from activations of the corresponding transmitters. The fixed time delay is also called a source excitation window. One can also vary this time delay for individual element. In this design the analog-to-digital converter (ADC) electronics is turned on at time T0 so that we can record source signatures at receivers near the edges when transmitters near the center are still in activation. One can also activate the ADC electronics at some other time if he/she is not interested in recording the source signatures. In FIG. 5, time t0 is the start of the first transmission and time ti is the start of the first reception.

### Part II: C-Wave Data Processing

Traditional beamforming of ultrasound data utilizes dynamic focusing method or pixel-based beamformers for focused beams, divergent beams, or planewaves [12-17]. The existing imaging method can't handle C-wave beam data. In this section we disclose a method of beamforming for C-wave ultrasound data.

### 2.1 Beamforming Formulation of C-Wave Data

An input data sample at time t and at receiver location xᵣ can originate from a scatter at an unknown position (x, z) illuminated by an incident wave from a transmitter at location xₛ. The travel time satisfies the following equation:

$$t ( {x}_{r} , x , z ) + t ( {x}_{s} , x , z ) = t + Δ {t}_{B} ( {x}_{s} ) \qquad ( 1 )$$

where t is the observed time of a reflection signal at the receiver xᵣ for a given beam. Δt_{B} is a transmitter time delay for this beam at location xₛ. (x, z) is the image (or scatter) position. t(xᵣ, x,z) is the travel time from xᵣ to (x, z), and t(xₛ, x,z) is the travel time from xₛ to (x, z).

The above equation defines an ellipse in the image domain, which is sometime called an impulse response curve for a transmitter and a receiver pair at a given travel time [18]. As the transmitter position xₛ moves away from the beam center location xc, the transmitter time delay Δt_{B} increases in C-wave beam data. That is, as xₛ changes, the ellipse in equation (1) changes in both foci positions and size. The envelope of all the ellipses forms an impulse response curve for an input sample of C-wave beam data. Please recall the input sample is collected when many transmitters are emitting simultaneously with certain time delays. The impulse response curve represents all possible spatial locations where one sample in one input beam contributes to the image formation. The final image is the summation of all impulse response curves for all time samples of all C-wave beams. This is the key concept of our method.

FIG. 6 illustrates the formation of a beam impulse response (thick curve) from a collection of single transmitter impulse responses for a given receiver and a given observation time. Each single transmitter impulse response is a trajectory in image domain on which Equation (1) is satisfied. The envelope of these single transmitter impulse responses is a trajectory in image domain where a data sample in an ultrasound beam effectively contributes to. This concept applies to all types of ultrasound beam data: focused beam, divergent beam, planewave beam, and any other beam configurations.

The key to our method is to find the envelope of all the ellipses as the transmitter position changes (while holding all other geometry parameters fixed). We define the family of curves for all the single transmitter ellipses as:

$$f ( x , z ; {x}_{s} ) = t ( {x}_{r} , x , z ) + t ( {x}_{s} , x , z ) - t - Δ {t}_{B} ( {x}_{s} ) \qquad ( 2 )$$

Its envelope, by definition, is given by:

$$f ( x , z ; {x}_{s} ) = 0 \qquad ( 3 a )$$

$$\frac{\partial f}{\partial {x}_{s}} ( x , z ; {x}_{s} ) = 0 \qquad ( 3 b )$$

The solution for (x, z) is then given by:

$$t ( {x}_{r} , x , z ) + t ( {x}_{s} , x , z ) = t + Δ {t}_{B} ( {x}_{s} ) \qquad ( 4 a )$$

$$\frac{\partial t ( {x}_{s} , x , z )}{\partial {x}_{s}} = \frac{\partial Δ {t}_{B} ( {x}_{s} )}{\partial {x}_{s}} \qquad ( 4 b )$$

Equation (4) gives a general formula for construction of an impulse response curve for one sample of an ultrasound beam data, including the C-wave beam data. The only requirement is that the transmitter delay function Δt_{B} (xₛ) be differentiable.

We will use C-wave beam data as an example, but our method equally applies to other type of ultrasound beam data.

For C-wave beam data acquisition we devise the following family of functions for the transmitter delays:

$$Δ {t}_{B} ( {x}_{s} ) = f ( {x}_{s} , R , β , γ , α ) \qquad ( 5 )$$

where R is the radius of a circle that defines the centers of all ellipses (FIG. 2, dash line). The bottom portion of the ellipse is the wavefront of a C-wave (FIG. 2, solid line from point A to point B). In equation (5) β is a scalar for the major axis of the ellipse. γ is a scalar for the minor axis of the ellipse, and α is the tilt angle of the C-wave wavefront. The center of the ellipse is at (x0, z0)=(R sin α,R cos α). The major radius is βR and minor radius is γR. The following equation completely describes the C-wave wavefront ellipse:

$$\frac{{[ ( x - {x}_{0} ) cos a - ( z - {z}_{0} ) sin α ]}^{2}}{{β}^{2} {R}^{2}} + \frac{{[ ( x - {x}_{0} ) sin α + ( z - {z}_{0} ) cos a ]}^{2}}{{γ}^{2} {R}^{2}} = 1 \qquad ( 6 )$$

which is shifted by (x0, z0) and rotated by the angle α. The distance between an element at (xₛ, zₛ) on the transducer and the ellipse in equation (6) is given by:

$$d ( {x}_{s} ) = \sqrt{{( {x}^{*} - {x}_{s} )}^{2} + {( {z}^{*} - {z}_{s} )}^{2}} \qquad ( 7 )$$

where (x*, z*) is a point on the ellipse whose distance to the element at (xₛ zₛ) is minimal. This point can be found by solving the following set of equations:

$${( \frac{( {x}^{*} - {x}_{0} )}{β R} cos a - \frac{( {z}^{*} - {z}_{0} )}{β R} sin α )}^{2} + {( \frac{( {x}^{*} - {x}_{0} )}{γ R} sin α + \frac{( {z}^{*} - {z}_{0} )}{γ R} cos a )}^{2} = 1 \qquad ( 8 a )$$

and

$$\frac{{z}^{*} - {z}_{s}}{( {x}^{*} - {x}_{s} )} = \frac{( {z}^{*} - {z}_{0} ) ( {β}^{- 2} {sin}^{2} α + {γ}^{- 2} {cos}^{2} a ) - ( {x}^{*} - {x}_{0} ) ( {β}^{- 2} - {γ}^{- 2} ) sin α cos α}{( {x}^{*} - {x}_{0} ) ( {β}^{- 2} {cos}^{2} a + {γ}^{- 2} {sin}^{2} α ) - ( {z}^{*} - {z}_{0} ) ( {β}^{- 2} - {γ}^{- 2} ) sin α cos α} \qquad ( 8 b )$$

For a given choice of the beam parameters (R, β, γ, α), one can solve the above two equations for (x*, z*) as a function of the element position (xₛ, xₛ). The transmitter time delay Δt_{B}(xₛ), for the element at xₛ, is then given by:

$$Δ {t}_{B} ( {x}_{s} ) = Δ {t}_{0} + \frac{d ( {x}_{s} ) - d ( {x}_{min} )}{V} \qquad ( 9 )$$

where V is a speed of sound used in setting up the transmitter time delay. xₘᵢₙ is a point on the transducer that has the minimum value of the equation (7). Δt₀ is the initial delay for this beam. It can be set to an arbitrary number as long as Δt_{B} (xₛ)≥0 is satisfied for all elements. We set Δt₀=0 for simplicity.

The solution to equations (8a) and (8b) can be expressed as:

$${x}^{*} = {x}_{0} + R {( β cos a cos φ + γ sin α sin φ )}^{} \qquad ( 10 a )$$

$$and$$

$${z}^{*} = {z}_{0} - R {( β sin a cos φ - γ cos α sin φ )}^{} \qquad ( 10 b )$$

where angle φ is determined by solving the following equation:

$$\frac{{z}_{0} - {z}_{s} - R ( β sin a cos φ - γ cos α sin φ )}{{x}_{0} - {x}_{s} + R ( β cos a cos φ + γ sin α sin φ )} = - \frac{( β sin a cos φ - γ cos α sin φ ) ( {β}^{- 2} {sin}^{2} α + {γ}^{- 2} {cos}^{2} a ) + ( β cos a cos φ + γ sin α sin φ ) ( {β}^{- 2} - {γ}^{- 2} ) sin α cos α )}{( β cos a cos φ + γ sin α sin φ ) ( {β}^{- 2} {cos}^{2} a + {γ}^{- 2} {sin}^{2} α ) + ( β sin a cos φ - γ cos α sin φ ) ( {β}^{- 2} - {γ}^{- 2} ) sin α cos α} \qquad ( 11 )$$

In the example below we use the following parameters: β=2, γ=1, −32°≤α≤+32°, and R=2.5 L, where L is the total aperture length of the transducer.

It is important to point out that, besides the ellipses in equation (6), other family of functions for the transmitter delays can be devised, such as circle, oval, banana, and Mexican hat, to name a few.

### 2.2 Implementation Details

The recommended implementation includes the following steps:
1. Take one input trace of a C-wave beam data at a given receiver location,
2. Perform necessary frequency filtering to protect the trace from aliasing or wavelet distortion during beamforming, if desired,
3. Spray the data samples of the trace along the impulse response curves calculated using equations (4), (5), (6), (7), (8), (9), (10) and (11). Also compute necessary attributes such as transmitter-receiver offset on the transducer, reflection angle at image point, wavelet stretch, anti-aliasing frequencies etc.
4. Accumulate image contributions, with options to form partial images for common image point gather generation.
5. Perform amplitude normalization for true reflection amplitude preservation, if required.
6. Repeat steps (1)-(5) for all input traces of all C-wave beams at all receiver locations.
7. Perform post processing and coherent compounding to obtain the final image.

The implementation method disclosed herein is robust and fast when analytical functions exist for both travel time calculation and time delay calculation. In the case where tissue sound speed varies spatially the method still yields quality images but requires a numerical solution to equation (4).

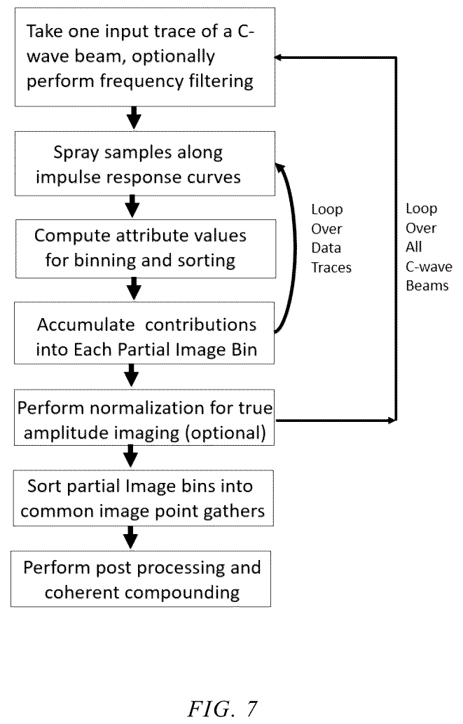

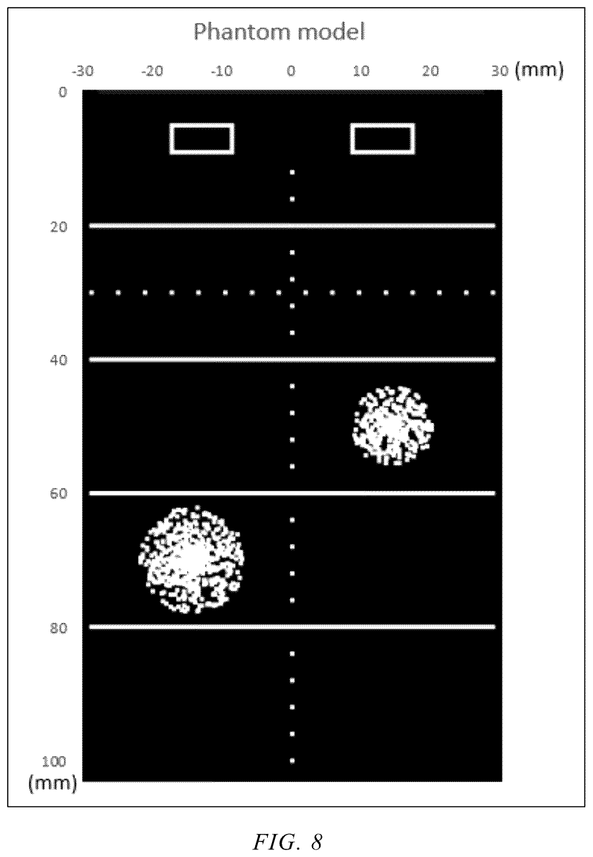

In the workflow diagram of the C-wave beamformer (FIG. 7) we have included the generation of partial image volumes and common image point gathers. The common image point gathers are useful for estimation of spatially varying effective sound speed values in order to produce the best ultrasound images. They are also useful for estimation of impedance and Poisson ratio properties from analysis of amplitude variation with reflection angles. They are even more useful for optimal compounding or stacking post beamforming. We have separate patent applications to cover all these aspects [19-21].

### Part III: Example

### 3.1 Echo Data Simulation

We use a modified version of Fresnel Simulator from Ultrasound Toolbox (USTB, https://www.ustb.co) for generation of numerical ultrasound beam data. The use of this simulator is subject to the citation rule. We sincerely thank the authors for making it available in the public domain [11]. The simulator is based on Fresnel approximation of diffraction of acoustic waves for rectangular transducers in a linear time invariant (LTI) system. Inputs to the simulator include a phantom model specification, a transducer specification, and a waveform specification. The phantom model used in this simulation contains:
two rectangular boxes with a depth range between 7-9 mm,
4 flat continuous reflectors at 20 mm, 40 mm, 60 mm and 80 mm depth,
A hyperechoic target with 8 mm radius at 70 mm depth and a second hyperechoic target with 6 mm radius at 50 mm depth,
A row of scatter points at 30 mm depth and a column of scatter points at the center of the model.

FIG. 8 is a depict of the phantom model. The transducer is a linear array with 256 elements (0.2 mm in pitch size) and each element has a width of 0.18 mm and a height of 5 mm. The central frequency of the simulated echo data is 6.25 MHz with 80% useful bandwidth and sampling frequency was 24 MHz.

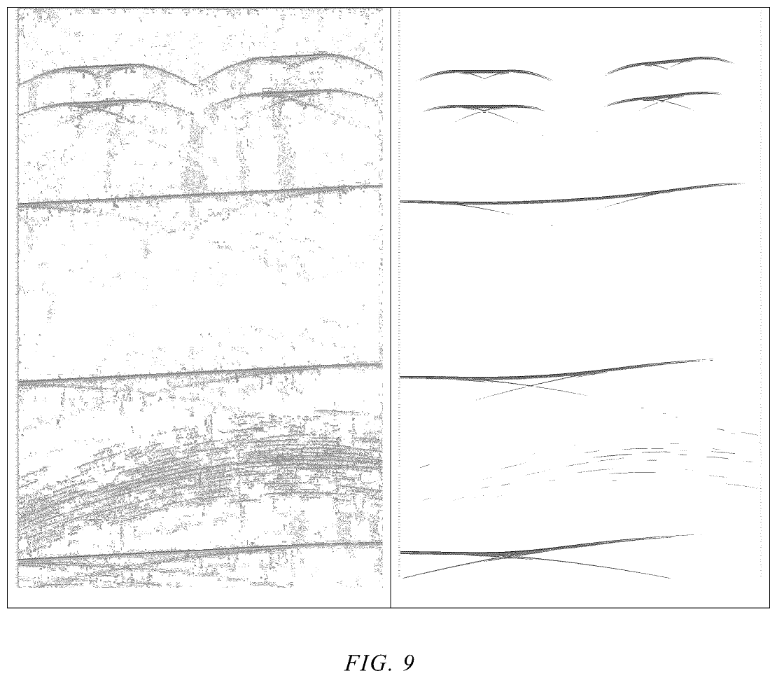

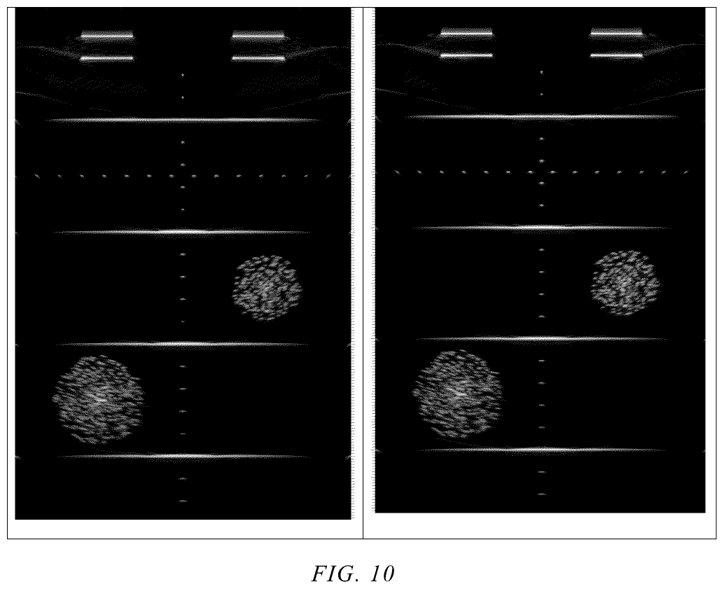

We have simulated 74 C-wave beams with tilt angles ranging from −32 to 32 degree as well as 74 planewave beams with tilt angles from −32 to 32 degree. The simulation time for a C-wave beam is the same as a planewave beam. FIG. 9 shows a planewave beam with a tilt angle of −5 degree (left) and a C-wave beam with a tilt angle of the same (right).

### 3.2 Resolution Test

FIG. 10 shows a comparison of an image of the 74 planewave beams (left) and another image of the 74 C-wave beams (right). All other parameters are the same. We see good resolution and quality on both images. In the central portion of the image domain the C-wave image is stronger because of the enhanced focusing effect of C-wave beams.

### 3.3 Signal-to-Noise Ratio Test

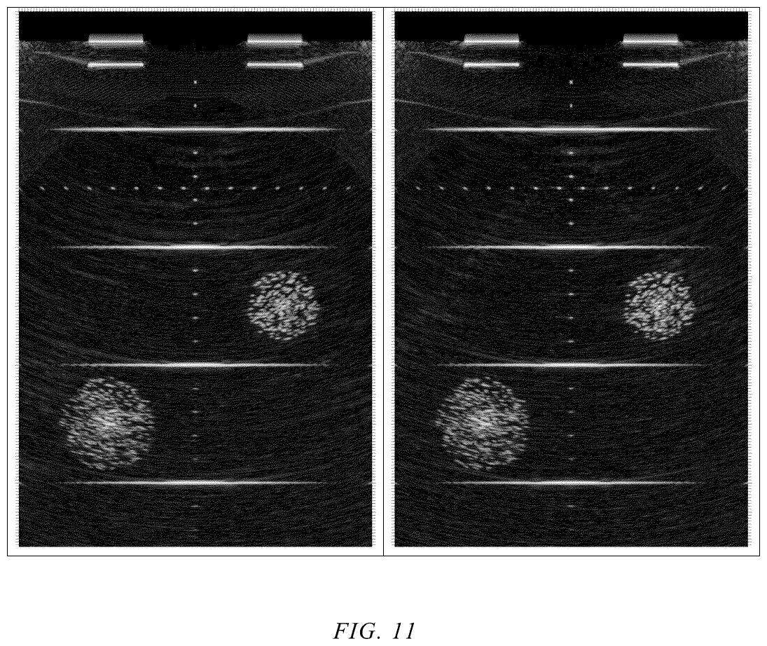

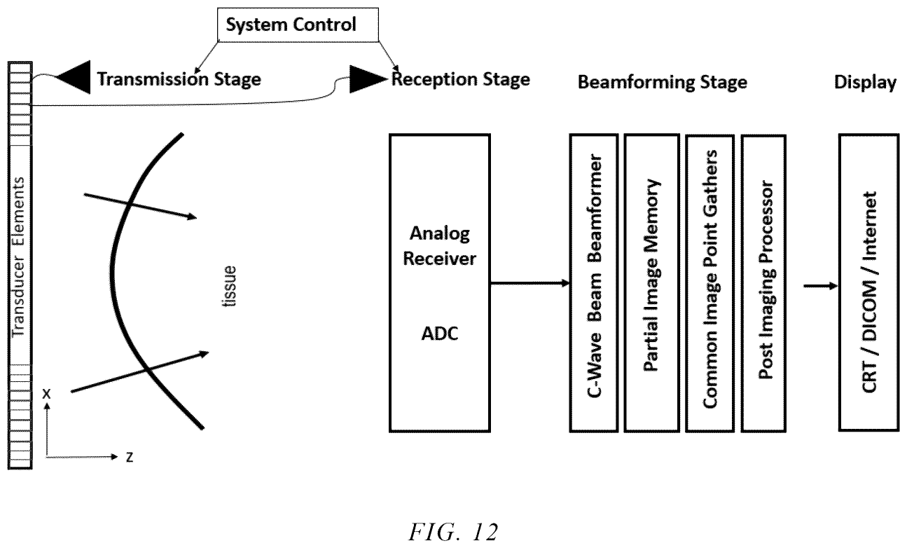

To test the impact of random data noises on image quality we add additive random noises whose maximum amplitudes are set at 30 dB while the maximum amplitudes of the original simulation data are scaled to 60 dB. The random noises are additive and have the same frequency band as the signals. FIG. 11 shows a comparison of an image of the 74 planewave beams (left) and an image of 74 C-wave beams (right), all with the same level of additive noises. We do see some speckles in the image when noises are added to the input but they almost have no impact on the image quality and resolution. When we increase the noise level the speckles become stronger and plenty. We believe some speckles seen on in-vivo ultrasound images are caused by noises in data acquisition and others are real diffractors in tissues and organs. Sometimes it is difficult to distinguish between the two.

FIG. 12 is a schematic representation of the C-Wave beam imaging architecture of one embodiment of the present invention. The processing unit contains one or more CPUs and one or more GPUs. One of the CPU sends instructions to the transmission and reception device to first transmit an acoustic pulse to each element of the transducer within a transmit aperture with a time delay that is specially designed for a C-wave beam, and then receive and record acoustic echoes reflected from tissue contrasts. The echo signals are sent to the processing unit for special processing and beamforming of C-wave beams on the CPUs, GPUs, or both. The final image is displayed on a local monitor or transmit via TCP/IP to a remote display device.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

### REFERENCES

[1] Richard S. C. Cobbold (2007), Foundations of Biomedical Ultrasound, Oxford University Press, pages 431-437.
[2] O. H. Schuck (1957), Variable Focus Transducer, U.S. Pat. No. 3,090,030, May 14, 1963.
[3] B. S. Hertzberg and W. D. Middleton (2016), Ultrasound: The Requisites, The Third Edition, Elsevier. Chapter 1, pages 3-31. Also at expertconsult.com.
[4] P. Suetens (2009), Fundamentals of Medical Imaging. 2nd Edition, Cambridge University Press, pages 33-158.
[5] D. M. Smith, D. F. Specht, L. V. Cabrea, K. D. Brewer, and D. J. Specht (2021), Concave Ultrasound Transducers and 3D Arrays, U.S. patent application Ser. No. 17/099,116, Filed Nov. 16, 2020.
[6] I. Waechter-Stehle and J. Weese (2020), 3D Ultrasound Imaging System, U.S. patent application Ser. No. 18/894,398, Filed Jun. 5, 2020.
[7] J. Flynn and R. E. Daigle (2019), Estimation and Display for Vector Doppler Imaging using Plane Wave Transmissions, U.S. patent application Ser. No. 16/560,851, Filed Sep. 4, 2019.
[8] T. K. Song and J. H. Chang (2003), Synthetic Aperture Focusing Method for Ultrasound Imaging Based on Planar Waves, U.S. patent application Ser. No. 10/180,614, Filed Jun. 25, 2002.
[9] N. T. Schiefler, J. M. Maia, F. K. Schneider, A. J. Zimbico, A. A. Assef, and E. T. Costa (2018), Generation and Analysis of Ultrasound Images Using Plane Wave and Sparse Arrays Techniques, Sensors, Vol. 18, 3660.
[10] G. Montaldo, M. Tanter, J. Bercoff, N. Benech, and M. Fink (2009), Coherent Planewave Compounding for very high Frame Rate Ultrasonography and Transient Elastography, IEEE Trans Ultrasonics, Ferroelectronics and Frequency Control, Vol. 56, pages 489-506.
[11] A. Rodriguez-Molares, Fresnel simulator, http://www.ustb.no/examples/fresnel/
[12] R. E. Daigle (2009), Ultrasound Imaging System with Pixel Oriented Processing, U.S. Patent 0112095 A1, May 19, 2009.
[13] R. Zemp and M. F. Insana (2007), Imaging with Unfocused Regions of Focused Ultrasound Beams, J. Acoust. So. Amer. Vol. 121, pages 1491-1498.
[14] N. Q. Nguyen and Richard Q. Prager (2016), High-resolution Ultrasound Imaging with Unified Pixel-Based Beamforming, IEEE Transactions on Medical Imaging, Vol. 35, pages 98-108.
[15] O. M. H. Rindal (2019), Software Beamforming in Medical Ultrasound Imaging—a Blessing and a Curse, Ph.D. Thesis, University of Oslo.
[16] O. M. H. Rindal, A. Rodriguez-Molares, and A. Austeng (2018), A Simple, Artifact-free, Virtual Source Model, IEEE International Ultrasonics Symposium, IUS 1-4. https:://doi.org/10.1109/ultsym.2018.8579944.
[17] D. J. Napolitano, B. D. DeBusschere, G. W. McLaughlin, L. Y. Mo, C. H. Chou, T. L. Ji, R. W. Steins (2011), Continuous Transmit Focusing Method and Apparatus for Ultrasound Imaging Systems, U.S. Pat. No. 8,002,705, Issued Aug, 2011.
[18] O. Yilmaz (2011), Seismic Data Analysis: Processing, Inversion and Interpretation of Seismic Data, Society of Exploration Geophysicists.
[19] C. Peng, and J. Tang (2021), Acquisition and Processing of V-wave Ultrasound Data Using a Linear or Curved Array Transducer, U.S. Patent Appl. No. 63/184,174, Filed May 4, 2021.
[20] C. Peng, and J. Tang (2021), Imaging Tissues and Organs Behind Bones Using an Ultrasound Array Transducer, U.S. Patent Appl No. 63/197,932, Filed Jun. 7, 2021.
[21] C. Peng, and J. Tang (2021), Ultrasound Imaging using Focusing Beams for Reducing Mechanical Index and Thermal Index, U.S. Patent Appl No. 63/243,325, Filed Sep. 13, 2021.

What is claimed is:

1. A method of acquiring ultrasound radio-frequency (RF) data using C-wave beams, comprising:
providing an ultrasound transducer, the ultrasound transducer including a plurality of elements acting as both transmitters and receivers;
transmitting sound waves from the transmitters of the ultrasound transducer within a transmit aperture with transmitter time delays being programed in such a way that sound waves are the C-wave beams that bend inward on both edges in a C shape; and
receiving the sound waves using the receivers of the ultrasound transducer,
wherein the coherent wavefront includes a variable tilt angle and a variable apex, and the variable apex moves away from a center of the ultrasound transducer as the variable tilt angle increases in absolute value;
wherein the variable apex is an acoustical energy focusing center of the coherent wave; and
wherein the variable tilt angle is an angle between a line connecting the center of an ellipse of the C-wave wavefront and the center of the ultrasound transducer and a vertical line passing the center of the ultrasound transducer.

2. The method of claim 1, wherein the ultrasound transducer is a linear array transducer, a curved array transducer, a phased array transducer, or a matrix array transducer.

3. The method of claim 1, wherein a first group of the elements of the ultrasound transducer transmit a first local coherent wave propagating in a first inward direction, a second group of elements of the ultrasound transducer transmits a second local coherent wave in a second inward direction; the first inward direction opposes the second inward direction; and the first local coherent wave and second local coherent wave combine to form the C-wave beams.

4. The method of claim 1, wherein the elements at both edges of the ultrasound transducer start transmission earlier than the elements at the center of the ultrasound transducer with a time slope that is a function of the variable tilt angle and the variable apex.

5. The method of claim 1, wherein the absolute value of the tilt angle is equal or greater than 0 and equal or less than a predefined positive number. The predefined positive number can be, for example, 20, 25, 30, 32, 34, 36, 38, 40, 45, 50, 55, 60, 65, 70, 75, 80, 85, or 90.

6. The method of claim 1, wherein the C-wave beams have a 3D bowl shape with two variable tilt angles and one variable apex.

7. The method of claim 1, further comprising:
(i) taking a trace from input data acquired using the C-wave beams;
(ii) optionally performing a frequency filtering to protect the trace from aliasing or excessive wavelet distortion during beamforming;
(iii) spraying the data samples of the trace along impulse response curves;
(iv) accumulating contributions at each image location, optionally forming partial image volumes for generation of common image point gathers;
(v) repeating steps (i)-(iv) for all traces in the data; and
(vi) performing post processing and coherent compounding to obtain a final image.

8. A system for acquiring and processing ultrasound radio-frequency (RF) data acquired using C-wave beams, comprising:
an ultrasound transducer, the ultrasound transducer including a plurality of elements;
a transmission and reception device;
a display device;
a keyboard;
a pointing device; and
a processing unit that contains a CPU (central processing unit) and a GPU (graphic processing unit),
wherein the CPU and the GPU are adapted to:
acquire, via the ultrasound transducer and the transmission and reception device, raw RF data using the C-wave beams;
process and send the raw RF data to CPU memories or GPU memories;
beamform the raw RF data on the CPU, the GPU, or both to obtain an ultrasound image;
process and send the ultrasound image to the display device;
display, via the display device, the ultrasound image; and
repeat the above steps for a next frame.

9. The system of claim 8, wherein the display device is connected to the processing unit remotely, via internet connection, wireless connection, or satellite connection.

10. The system of claim 8, wherein the ultrasound transducer is a linear array transducer, a curved array transducer, a phased array transducer, or a matrix array transducer.

11. The system of claim 8, wherein the keyboard is a wireless keyboard or a software keyboard installed on the processing unit.

12. The system of claim 8, wherein the transmission and reception device is programmed to transmit and receive various types of the C-Wave beams.

13. The system of claim 8, wherein the pointing device is a touch screen.

14. The system of claim 8, wherein using the C-wave beams comprising:
providing an ultrasound transducer, the ultrasound transducer including a plurality of elements acting as both transmitters and receivers;
transmitting sound waves from the transmitters of the ultrasound transducer within a transmit aperture with transmitter time delays being programed in such a way that sound waves are the C-wave beams that bend inward on both edges in a C shape; and
receiving the sound waves using the receivers of the ultrasound transducer,
wherein the coherent wavefront includes a variable tilt angle and a variable apex, and the variable apex moves away from a center of the ultrasound transducer as the variable tilt angle increases in absolute value;
wherein the variable apex is an acoustical energy focusing center of the coherent wave; and
wherein the variable tilt angle is an angle between a line connecting the center of an ellipse of the C-wave wavefront and the center of the ultrasound transducer and a vertical line passing the center of the ultrasound transducer.

15. The system of claim 14, wherein the C-wave beams have a 3D bowl shape with two variable tilt angles and one variable apex.
